(19) United States
(12) Patent Application Publication
(10) Pub. No.: US 2010/0316585 A1
(43) Pub. Date: Dec. 16, 2010

(54) Use of Magnesium Stearate Dihydrate for Lubrication of Solid Industrial or Consumer Products

(76) Inventors: Stephen H. Wu, Chesterfield, MO (US); Brian K. Cheng, Chesterfield, MO (US); Gary A. Nichols, Wildwood, MO (US); Jae H. Park, Olivette, MO (US)

Correspondence Address: Mallinckrodt Inc., 675 McDonnell Boulevard, HAZELWOOD, MO, 63042, US

(21) Appl. No.: 12/866,261

(22) PCT Filed: Feb. 11, 2009
(86) PCT No.: PCT/US09/33705

§ 371 Date: Aug. 5, 2010

Related U.S. Application Data
(60) Provisional application No. 61/027,588, filed on Mar. 12, 2008.
(60) Provisional application No. 61/042,001, filed on Apr. 3, 2008.
(60) Provisional application No. 61/055,157, filed on May 22, 2008.
(60) Provisional application No. 61/027,580, filed on Mar. 11, 2008.

(51) Int. Cl. A61K 8/18; A23L 1/00; A61K 47/00; A61Q 1/02; A61Q 1/08; A61Q 1/10; C09D 201/00; C08K 5/09

(52) U.S. Cl. 424/63; 426/531; 426/618; 426/658; 514/785; 106/243; 524/400

(57) ABSTRACT

The invention provides lubricant compositions comprising magnesium stearate dihydrate. The lubricant compositions may be used to lubricate a variety of solid materials that are used in industrial or consumer products.

### FIELD OF THE INVENTION

The invention generally relates to lubricant compositions and methods for lubricating solid materials. In particular, the invention provides magnesium stearate dihydrate compositions that may be used to lubricate solid industrial or consumer products.

### BACKGROUND OF THE INVENTION

Lubricants are widely used in powder blending applications for their anti-adherent activity (i.e., prevent sticking to punch faces and die walls), glidant activity (i.e., improve the flowability of the powder or granules), and lubricant activity (i.e., reduce friction, transfer heat, and prevent corrosion during the process). Magnesium stearate (MgSt) is widely used as a lubricant in the manufacture of tablets or capsules, food products, cosmetic products, and industrial products. MgSt has advantages over other lubricants because of its high melting temperature, high lubricity at a low concentration, large covering potential, general acceptance as safe, nontoxicity, and its excellent stability profile.

Magnesium stearate is commercially available mainly in the monohydrate form (MgSt-M) or as a mixture of the monohydrate along with trace amounts of other crystalline forms, such as the dihydrate (MgSt-D) and trihydrate, and amorphous forms. The composition of MgSt preparations not only varies from manufacturer to manufacturer, but also from lot-to-lot. Thus, variations in the composition of MgSt preparations and the different crystalline states of the various hydrate forms could affect the uniformity of the ingredients blended together, as well as the quality of the resulting product. Because of the variations in the compositions of MgSt preparations, there is a need for pure forms of MgSt. Furthermore, there is a need for methods of using pure MgSt-D as a lubricant in consumer and industrial products.

### SUMMARY OF THE INVENTION

One aspect of the present invention provides a method for lubrication of a solid material. The method comprises combining the solid material with a lubricant composition comprising at least 40% by weight of magnesium stearate dihydrate.

Another aspect of the invention encompasses a food product. The food product comprises an edible material and a lubricant composition comprising at least 40% by weight of magnesium stearate dihydrate.

Yet another aspect of the invention provides a dry powder cosmetic product. The dry powder cosmetic product comprises a dry powder phase, a liquid binder phase, and a lubricant composition comprising at least 40% by weight of magnesium stearate dihydrate.

An additional aspect of the invention encompasses a dry paint product. The dry paint product comprises a dry paint powder and a lubricant composition comprising at least 40% by weight of magnesium stearate dihydrate.

Other features and iterations of the invention are described in more detail below.

### DESCRIPTION OF THE FIGURES

FIG. 1 presents the powder X-ray diffraction patterns of the monohydrate and dihydrate forms of magnesium stearate (MgSt).

FIG. 2 presents scanning electron micrographs of MgSt-monohydrate (A) and MgSt-dihydrate (B).

FIG. 3 presents the differential scanning calorimetry profiles of the monohydrate and dihydrate forms of MgSt.

FIG. 4 shows the thermogravimetric analysis for the monohydrate and dihydrate forms of MgSt.

FIG. 5 presents the near-infrared spectra of the monohydrate and dihydrate forms of MgSt.

FIG. 6 presents an in-line effusivity plot of (four) baseline runs with neat microcrystalline cellulose.

FIG. 7 presents an effusivity plot of Batch 7 (acetaminophen-microcrystalline cellulose-dibasic calcium phosphate (APAP-MCC-DCP) lubricated with 1% MgSt-M).

FIG. 8 presents an effusivity plot of Batch 11 (APAP-MCC-DCP lubricated with 1% MgSt-D).

FIG. 9 presents an effusivity plot of Batch 12 (APAP-MCC-lactose monohydrate (APAP-MCC-LAC) lubricated with 1% MgSt-M).

FIG. 10 presents an effusivity plot of Batch 14 (APAP-MCC-LAC lubricated with 1% MgSt-D).

FIG. 11 presents a scatterplot analysis of the average compression coefficient 50 mm/s as a function of blend time for the monohydrate and dihydrate forms of MgSt.

FIG. 12 presents a scatterplot analysis of the standard deviation of compression as a function of blend time for the monohydrate and dihydrate forms of MgSt.

FIG. 13 is a main effects plot for effusivity as a function of MgSt type and percent of MgSt.

FIG. 14 is a main effects plot for total compression forces as a function of MgSt type and percent of MgSt.

FIG. 15 is a main effects plot for ejection force as a function of MgSt type and percent of MgSt.

FIG. 16 plots the % compressibility of blends comprising starch as a diluent. MG1 refers to MgSt-M and MG2 refers to MgSt-D.

FIG. 17 presents dissolution profiles for APAP-MCC-DCP blends with 0.3% MgSt.

FIG. 18 presents dissolution profiles for APAP-MCC-DCP blends with 1.0% MgSt.

FIG. 19 presents dissolution profiles for APAP-MCC-LAC blends with 1.0% MgSt.

### DETAILED DESCRIPTION

The present invention provides lubricant compositions and methods of using the lubricant compositions to lubricate solid materials. Typically, the lubricant composition comprises at least 40% by weight MgSt-D. In particular, it has been discovered, as illustrated in the examples, that MgSt-D provides certain advantages as a lubricant when compared to MgSt-M. For example, use of MgSt-D for pharmaceutical applications generally achieves comparable blend uniformity of the pharmaceutically active ingredients and excipients in a shorter blending time compared to MgSt-M. Additionally, the blend uniformity is typically less sensitive to blending time, and the mixture generally exhibits improved lubricating efficiency in subsequent tableting processes when MgSt-D is used as a lubricant compared to the use of MgSt-M. Furthermore, MgSt-D exhibits a stronger binding force than MgSt-M to the surfaces of powder particles, and MgSt-D imparts a strongly adhered water-repellent barrier to the particle surfaces. In view of the desirable properties of MgSt-D, it may be beneficially used in several industrial and consumer products as a lubricant.

### I. Lubricant Compositions

The lubricant compositions comprise MgSt-D. The amount of MgSt-D comprising the lubricant composition can and will vary depending upon the application. Typically, the lubricant composition will include at least 40% by weight of MgSt-D. In other embodiments, the lubricant composition will include at least 45%, 50%, 55%, 60%, 65%, 70%, 75%, 80%, 85%, 90%, 95%, 97% or greater than 99% by weight of MgSt-D. In an exemplary embodiment, the lubricant composition will comprise greater than 90% by weight of MgSt-D. In each of the foregoing embodiments, the lubricant composition typically will have less than about 5% by weight of MgSt-M. More typically, the lubricant composition will have less than about 4.5%, 4.0%, 3.5%, 3.0%, 2.5%, 2.0%, 1.5%, 1.0%, or less than about 0.5% by weight of MgSt-M. In an exemplary embodiment, the amount of MgSt-M is less than about 1.0% by weight.

Generally speaking, MgSt-D is composed of a mixture of stearic acid, palmitic acid, and water. The weight ratio of stearic acid, palmitic acid, and water can and will vary depending upon the manner in which the MgSt-D is made. In one embodiment, the weight ratio of stearic acid to palmitic acid may range from about 2:1 to 1:2. In an exemplary embodiment, the weight ratio of stearic acid to palmitic acid is about 2:1. MgSt-D having a weight ratio of stearic acid to palmitic acid of 2:1 may be manufactured according to the following two-step general scheme:

C₁₇H₃₅COOH+NaOH→C₁₇H₃₅COONa+H₂O; and

2C₁₇H₃₅COONa+MgSO₄.7H₂O→Mg(C₁₇H₃₅COO)₂.2H₂O+Na₂SO₄+6H₂O

Highly pure MgSt-D that is a crystalline form of matter and more particularly, is a stable polymorph may be manufactured using the reaction scheme detailed above in combination with the reaction conditions reported in U.S. Application Publication Nos. 2006/0281937 and 2006/0247456, both of which are incorporated herein by reference in their entirety.

As will be appreciated by a skilled artisan, the particle size of the MgSt-D can and will vary depending upon the solid material to be lubricated. The MgSt-D will, however, at least be generally the same particle size or smaller than the particle size of the solid material. In certain embodiments, the average diameter of the MgSt-D may range from about 1 to about 500 microns. In other embodiments, the average diameter of the MgSt-D may range from about 5 to about 250 microns. In another embodiment, the average diameter of the MgSt-D may range from about 5 to about 100 microns. Alternatively, the average diameter of MgSt-D may range from about 10 to about 50 microns. In other embodiments, the average diameter of the MgSt-D will be less than about 30 microns, less than about 25 microns, less than about 20 microns, less than about 15 microns, or less than about 10 microns.

In an exemplary embodiment, the MgSt-D particles may be less than about 30 microns and may have a D₅₀ (i.e., 50th percentile of the particle size distribution) of about 11 to about 16 microns, a D₉₀ (i.e., 90th percentile of the particle size distribution) of about 22 to about 28 microns, and a surface area of about 4.0 to about 7.5 m²/g depending upon particle size. In another exemplary embodiment, the MgSt-D particles may be micronized, i.e., they are reduced to less than 10 microns in diameter by conventional milling processes. These micronized particles have a D₅₀ of about 5 microns, a D₉₀ of less than about 10 microns, and a surface area of about 10 to about 20 m²/g depending upon the particle size.

Typically, the lubricant compositions may be suitably employed to lubricate a wide variety of solid materials irrespective of their form or size. For example, the lubricant composition may be used to lubricate a solid surface not having a reduced particle size such as a glass surface, metal surface, clay surface, ceramic surface, or a plastic surface. Alternatively, the lubricant compositions may be employed to lubricate solid materials having a reduced particle size. Non-limiting examples of solid materials having reduced particle sizes include powders, beads, granules, crystals, and encapsulated materials (e.g., lyophilized liposomes, encapsulated liquids, encapsulated semisolids, or encapsulated solids).

### II. Lubrication of Solid Materials

The lubricant compositions may be utilized to lubricate solid materials that form industrial or consumer products. Examples of industrial or consumer products include cosmetic products, food products, nutrition products, nutraceuticals, mineral products, paint products, toners, and powder coatings. The solid material may be a powder, a bead, a granule, a crystal, a particle, a flake, an encapsulated liquid, an encapsulated semi-solid material, an encapsulated solid material, a food particle, and the like. In general, the solid material is lubricated by combining the solid material with a lubricant composition of the invention.

The industrial or consumer product will generally comprise from about 0.1% to about 20% by weight of the lubricant composition comprising MgSt-D. In another embodiment, the industrial or consumer product may comprise from about 1% to about 10% by weight of the lubricant composition comprising MgSt-D. In a further embodiment, the industrial or consumer product may comprise from about 1% to about 2% by weight of the lubricant composition comprising MgSt-D.

a. Food or Nutrition Products

A variety of food or nutrition products may be contacted with the lubricant compositions of the invention. In one embodiment, the lubricant compositions may be used as anti-caking agents in dry powder food products. Examples of suitable food products that may be lubricated with the lubricant compositions include salts (e.g., sodium chloride, potassium chloride, garlic salt, onion salt, and the like), sugars (e.g., superfine sugar, powdered sugar, confectionary's sugar, icing sugar, and so forth), flours (e.g., cake flour, pastry flour, wheat flour, chickpea flour, rice flour, etc.), starches (e.g., corn starch, tapioca starch, and so forth), leavening agents (e.g., baking powder, baking soda, cream of tartar, and the like), and dry blend mixes (e.g., cake mixes, muffin mixes, bread mixes, quick bread mixes, cookie mixes, pudding mixes, biscuit mixes, pancake mixes, icing mixes, dry milk, cocoa mixes, dry coffee creamers, breakfast drink mixes, fruit-flavored drink mixes, energy drink mixes, sports drink mixes, weight management drink mixes, salad dressing dry mixes, dry soup mixes, seasoning mixes, and so forth).

The concentration of the lubricant composition may range from about 0.1% to about 10% by weight of the total weight of the food or nutrition product. More typically, the concentration of the lubricant composition will be about 1% to about 2% by weight of the total weight of the food product.

In another embodiment, the lubricant compositions may also be used as coating agents to extend the shelf life of food products or to adhere flavoring agents to food products. Non-limiting examples of suitable food or nutrition products including cereals, cereal-based products, crackers, cookies, pretzels, potato chips, tortilla chips, nuts, snack mixes, popcorn, cheese puffs, pork rinds, beef jerky, trail mix, granola, granola bars, breakfast bars, energy bars, etc. The food product may be contacted with the lubricant compositions in either batch or continuous processes; and the lubricant compositions may be sprayed or applied by other means well known in the art. The concentration of the lubricant composition may range from about 2% to about 8% by weight of the total weight of the coated food product.

b. Cosmetic Products

The lubricant compositions of the invention may also be used as dry binders or lubricants in dry powder cosmetic formulations or dry powder personal care formulations. Examples of dry powder cosmetics include dry foundation, face powder, wet/dry powder, pressed powder, loose powder, blush powder, rouge, eyelid powder, eye shadow, eyebrow pencil, eyeliner pencil, and the like. Examples of dry powder personal care formulations include solid deodorant, solid antiperspirant, dry pre-shave formulations, and so forth. In general, the compositions of the present invention may impart an unctuous feel and facilitate adherence of the formulation to the skin.

Typically, dry powder cosmetic formulations comprise a dry powder phase and a liquid binder phase. The dry powder phase may comprise a filler or extender such as a mineral silicate (e.g., silica, mica, talc, and the like), starch, cellulose, bentonite, hectorite, kaolin, chalk, diatomaceous earth, attapugite, zinc oxide, titanium dioxide, precipitated calcium carbonate, magnesium carbonate, calcium phosphate, synthetic polymer powder (e.g., polyethylenes, polyamides, polyesters, nylons, acrylates, acrylate copolymers, methacrylate copolymers, fluorinated polymers, etc.), and/or silicone resin powders/particles. The filler or extender may have a form of a particle, a spherical particle, or a flake. Typically, the diameter of a particle or flake will range from about 2 to about 500 microns, and preferably from 5 to about 50 microns. The thickness of a flake may range from about 0.1 to about 5 microns, and preferably from about 0.2 to about 3 microns.

The dry powder phase may further comprise at least one pigment. Examples of suitable pigments include white pigments (e.g., titanium oxide, zinc oxide, zirconium oxide, etc.), color pigments (e.g., red iron oxide, yellow iron oxide, black iron oxide, ultramarine blue, Berlin blue, chromium oxide, chromium hydroxide, carbon black, coal tar coloring material, D&C Red Nos. 6, 7, 9, 19, 21, 27, 40, D&C Orange Nos. 4, 5, 10, D&C Yellow Nos. 5, 13, 19, D&C Blue No. 1, natural coloring matter, and the like), and/or pearlescent pigments (e.g., fish scale guanine, mica titanium, bismuth oxychloride, and so forth).

The dry powder phase may also comprise an inorganic salt such as calcium carbonate, calcium chloride, calcium phosphate, calcium silicate, magnesium carbonate, aluminum silicate, magnesium silicate, and combinations thereof. Other ingredients that may be included in the dry powder phase include a sunscreen (e.g., octyl methoxycinnamate, oxybenzone, etc.), an antioxidant (e.g., alpha hydroxy acid, ascorbyl palmitate, grape seed extract, green tea extract, resveratrol, vitamins A, B, C, E, and so forth), a preservative (e.g., benzoyl peroxide, boric acid, EDTA, parabens, etc.) and other beneficial agents (e.g., allantoin, amino acids such as glycine, lysine, proline, or tyrosine, collagen, lanolin, lecithin, retinol, and the like).

The liquid binder phase of the dry powder cosmetic formulation may comprise oils, hydrocarbons, liquid synthetic esters, silicone oils, silicone emulsifiers, waxes, and the like. Exemplary liquid binders include cetyl alcohol, alcohol SD-40, beeswax, glycerin, polybutene, propylene glycol. Those skilled in the art will appreciate that the ratio of dry powder phase to liquid binder phase can and will vary depending upon the desired use of the formulation. Typically, the dry powder phase may comprise from about 80% to about 99% by weight of the total formulation and the liquid binder phase may comprise from about 1% to about 20% by weight of the total formulation.

Dry powder personal care formulations typically also comprise fillers, extenders, pigments, and liquid binders as detailed above. Deodorants and antiperspirants, however, also comprise an active ingredient, such as aluminum chloride, aluminum chlorohydrate, aluminum zirconium trichlorohydrate glycine, or aluminum hydroxybromide.

The concentration of the lubricant composition in the dry powder cosmetic or dry powder personal care formulation may range from about 1% to about 15% by weight of the total weight of the formulation, and more preferably from about 2% to about 8% by weight of the total weight of the formulation.

c. Paint Products

The lubricant compositions of the invention may also be used in the lubrication of dry paint products. For instance, a paint powder may be contacted with the lubricant composition of the invention. Paint powder, also referred to as powder coatings, may generally be thermosetting or thermoplastic. Thermosetting powder coatings typically comprise a cross-linker. In general, powder coatings may have a glass transition temperature (TG) of greater than 40° C., although a TG of less than 40° C. is possible in certain embodiments.

Generally speaking, paint powder comprises a polymer. Paint powder may also comprise pigments, hardeners, or other additives described in more detail below. The most common polymers that may be used include polyester, polyester-epoxy, straight epoxy and acrylics. The polymers may also be polyether or polyurethane, and the polymer may contain functional groups such as hydroxyl, carboxylic acid, carbamate, isocyanate, epoxy, amide and carboxylate functional groups.

The use in powder coatings of acrylic, polyester, polyether and polyurethane polymers having hydroxyl functionality is known in the art. Monomers for the synthesis of such polymers are typically chosen so that the resulting polymers have a TG greater than 50° C. Examples of such polymers are described in U.S. Pat. No. 5,646,228, which is hereby incorporated by reference in its entirety.

Acrylic polymers and polyester polymers having carboxylic acid functionality are also suitable for powder coatings. Monomers for the synthesis of acrylic polymers having carboxylic acid functionality are typically chosen such that the resulting acrylic polymer has a TG greater than 40° C., and for the synthesis of the polyester polymers having carboxylic acid functionality such that the resulting polyester polymer has a TG greater than 50° C. Examples of carboxylic acid group-containing acrylic polymers are described in U.S. Pat. No. 5,214,101, which is hereby incorporated by reference in its entirety. Examples of carboxylic acid group-containing polyester polymers are described in U.S. Pat. No. 4,801,680, which is hereby incorporated by reference in its entirety.

Also useful in the present powder coating compositions are acrylic, polyester and polyurethane polymers containing carbamate functional groups. Examples are described in WO Publication No. 94/10213, which is hereby incorporated by reference in its entirety. Monomers for the synthesis of such polymers are typically chosen so that the resulting polymer has a high TG, that is, a TG greater than 40° C. The TG of the polymers described above can be determined by differential scanning calorimetry (DSC).

Powder coatings may also comprise suitable curing agents. Non-limiting examples may include blocked isocyanates, polyepoxides, polyacids, polyols, anhydrides, polyamines, aminoplasts and phenoplasts. One skilled in the art will be able to select the appropriate curing agent, depending on the polymer used.

The polymer described above is generally present in the powder coatings of the invention in an amount greater than about 50 weight percent, such as greater than about 60 weight percent, and less than or equal to 95 weight percent, with weight percent being based on the total weight of the composition. For example, the weight percent of polymer can be between 50 and 95 weight percent. When a curing agent is used, it is generally present in an amount of up to 30 weight percent; this weight percent is also based on the total weight of the coating composition.

The powder coating compositions of the present invention may optionally contain other additives such as waxes for flow and wetting, flow control agents, such as poly(2-ethylhexyl)acrylate, degassing additives such as benzoin and microcrystalline waxes, MicroWax C, adjuvant resin to modify and optimize coating properties, antioxidants, ultraviolet (UV) light absorbers, fine particles of silica, fumed silica both treated and untreated, finely divided aluminum oxide, feldspar, calcium silicate, and catalysts. Examples of useful antioxidants and UV light absorbers include those available commercially from Ciba Specialty Chemicals Corporation under the trademarks IRGANOX and TINUVIN. These optional additives, when used, can be present in amounts up to 20 percent by weight, based on total weight of the coating.

In some embodiments of the invention, the lubricating composition may comprise from about 0.1% of the powder coating to about 20% of the powder coating. In other embodiments, the lubricating composition may comprise from about 2% to about 10% of the powder coating. For instance, the lubricant composition may comprise about 2%, about 3%, about 4%, about 5%, about 6%, about 7%, about 8%, about 9%, or about 10% of the powder coating. In certain embodiments, the lubricant composition may comprise from about 2% to about 6% of the powder coating.

The lubricating composition of the present invention, as well as any additional additives, can be added at any time during the formulation of the powder coating. For example, powder-coating compositions of the present invention may be prepared by first dry blending a polymer, and adding any suitable additives including a lubricating composition of the invention, in a blender, such as a Henschel blade blender. The blender is operated for a period of time sufficient to result in a homogenous dry blend of the materials. The blend may then be melt blended in an extruder, such as a twin-screw co-rotating extruder, operated within a temperature range sufficient to melt but not gel the components. The melt-blended powder coating composition may typically be milled to an average particle size of from, for example, 15 to 80 microns. Other methods known in the art for preparing powder coatings can also be used.

Powder coating compositions are most often applied by spraying, and in the case of a metal substrate, by electrostatic spraying, or by the use of a fluidized bed. Electrostatic spraying is generally performed by an electrostatic spray gun that consists essentially of a tube to carry airborne powder to an orifice with an electrode located at the orifice. The electrode is connected to a high-voltage (about 5-100 kv), low-amperage power supply. As the powder particles come out of the orifice they pass through a cloud of ions, called a corona and pick up a negative or positive electrostatic charge. The object to be coated is electrically grounded. The difference in potential attracts the powder particles to the surface of the part. They are attracted most strongly to areas that are not already covered, forming a reasonably uniform layer of powder even on irregularly shaped objects. The particles cling to the surface strongly enough and tong enough for the object to be conveyed to a baking oven, where the powder particles fuse to form a continuous film, flow, and optionally cross-linked.

The powder coating may be applied in a single sweep or in several passes to provide a film having a final thickness of from about 1 to about 10 mils (about 0.0254 mm to about 0.254 mm), usually about 2 to about 4 mils (about 0.0508 mm to about 0.1016 mm). Other standard methods for coating application can be employed such as brushing, dipping or flowing.

Generally, after application of the coating composition, the coated substrate is baked at a temperature sufficient to cure the coating. Metallic substrates with powder coatings are typically cured at a temperature ranging from 230° F. to 650° F. for about 30 seconds to about 30 minutes.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Examples 1-3

### Solid-State Characterization of MgSt Hydrates

Magnesium stearate (MgSt) is widely used as a lubricant in the pharmaceutical and nutraceutical industry. MgSt is commercially available mainly in the monohydrate form (MgSt-M) or as a mixture of the monohydrate with trace amounts of the dihydrate (MgSt-D) and amorphous forms. The physicochemical properties of these two forms were examined. The following three examples detail these analyses using pure dihydrate and monohydrate forms of MgSt (derived from a vegetable source) obtained from Mallinckrodt (Hazelwood, Mo.).

### Example 1

### Particle Size and Particle Properties of the Hydrates

Particle-size distributions were determined using a laser diffraction system (series 2600, Malvern Instruments Ltd., Malvern, UK) equipped with a 63-mm lens (size range of 1.2-118 μm) and a stirred cell. Particle size, percentage of water, and concentration of neat MgSt-M and MgSt-D are presented in Table 1. The neat dihydrate had a concentration of 95.4%, and the neat monohydrate had a concentration of 92.0%. The results also revealed that the nominal mean particle size of the monohydrate was 10.6 μm, whereas the dihydrate had a nominal mean particle size of 14.3 μm. In addition, the percent bound moisture was 2.8% for MgSt-M and 5.6% for MgSt-D. Although the percent of free water was generally low, MgSt-M had 0.6%, while little or no free water was found in MgSt-D.

TABLE 1
MgSt Pseudopolymorph Particle Size and Percent Water.

| | Mono- | Di- | Particle | Free | Monohydrate | Dihydrate |
|---|---|---|---|---|---|---|
| | hydrate | hydrate | size | water | water | water |
| Form | (%) | (%) | (μm) | (%) | (%) | (%) |
| MgSt-M | 92.0 | 0 | 10.6 | 0.6 | 2.8 | 0 |
| MqSt-D | 0 | 95.4 | 14.3 | 0 | 0 | 5.6 |

### Example 2

### Molecular Properties of the Hydrates

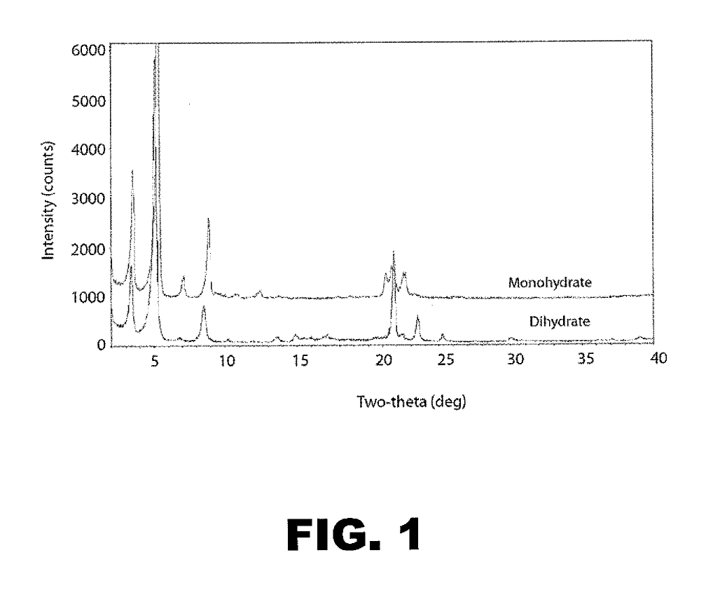

Powder X-ray diffraction patterns of the samples were measured with a Siemens D500 X-ray diffractometer over the range of 2q=2° to 40° and a 0.02° step size. FIG. 1 presents the PXRD analyses of the MgSt hydrates.

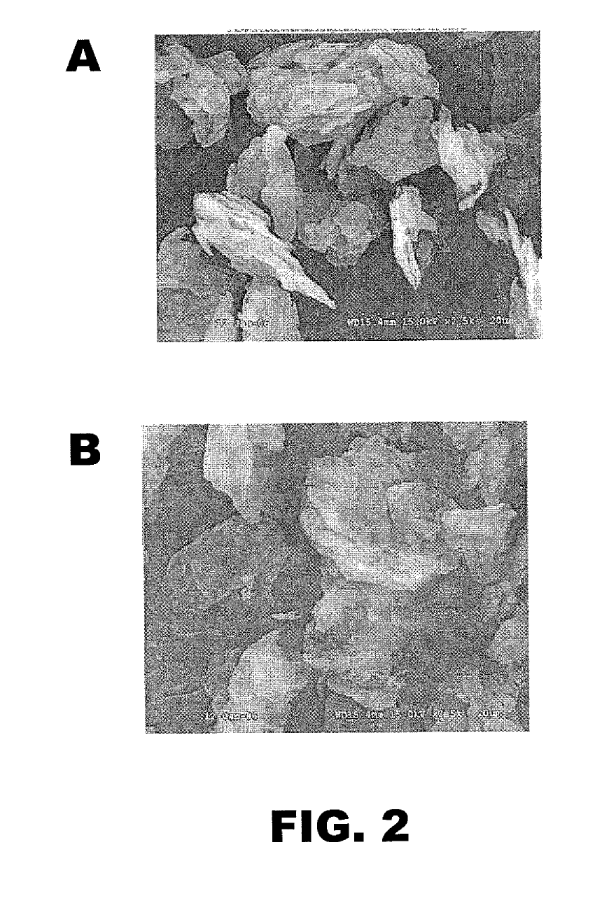

The morphologies of the samples were investigated using a scanning electron microscope (model S-4500, Hitachi, Japan). A Cressington 108 Auto/SE sputter coater was used (Cressington Scientific, Watford, UK). Images were captured with the secondary electron detector. A small portion of the powdered sample was distributed onto a conductive carbon adhesive disk on SEM stubs for SEM imaging. The specimens were sputter coated with gold-palladium to impart conductivity. The instrumental parameters were: electron beam source=W filament; accelerating voltages=15 kV; objective aperture=50 μm (aperture #3); vacuum mode=high vacuum; imaging detector(s): SE; magnification=1000×, 2500×, and 3500×; specimen tilt=0.0°; and working distance=nominal 16. FIG. 2 presents SEM micrographs of the MgSt hydrates at a magnification of 2500×.

### Example 3

### Thermal Properties of the Hydrates

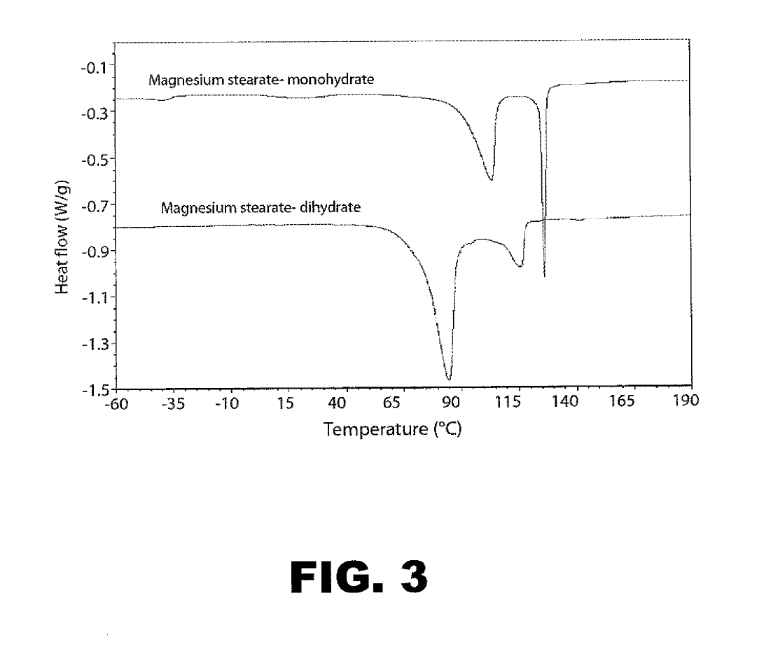

Differential scanning calorimetry (Q100 DSC, TA Instruments, New Castle, Del.) was done between temperatures from −60 to +190° C. at a heating rate of 2° C./min and a nitrogen purge of 50 mL/min. Samples (3-5 mg) were tested in crimped-aluminum pans, and an empty pan was used as a reference. The temperature axis and cell constant of DSC were previously calibrated with pure standard of indium. Data acquisition and analysis were conducted using TA Instruments software. DSC profiles of the MgSt hydrates are presented in FIG. 3.

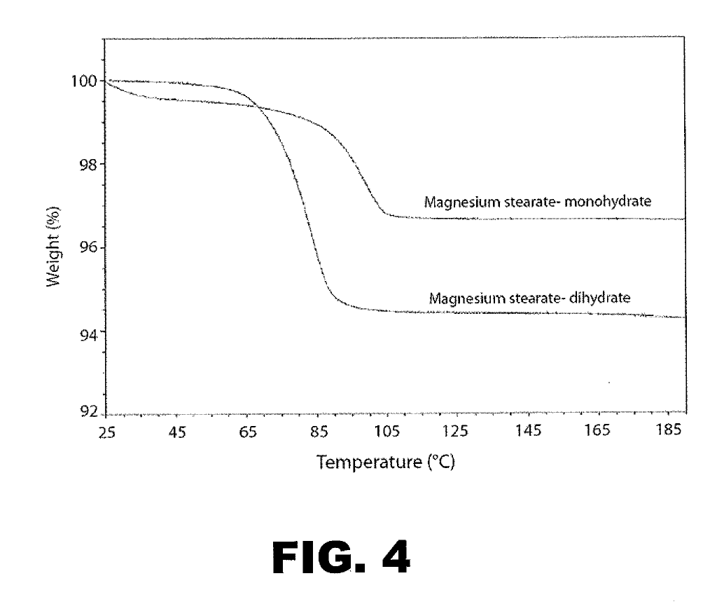

Thermal gravimetric analysis (Q50 TGA, TA Instruments) was carried out from 25 to 190° C. at a heating rate of 5° C./min. Data acquisition and analysis were conducted using TA Instruments software. FIG. 4 presents the TGA of MgSt-M and MgSt-D.

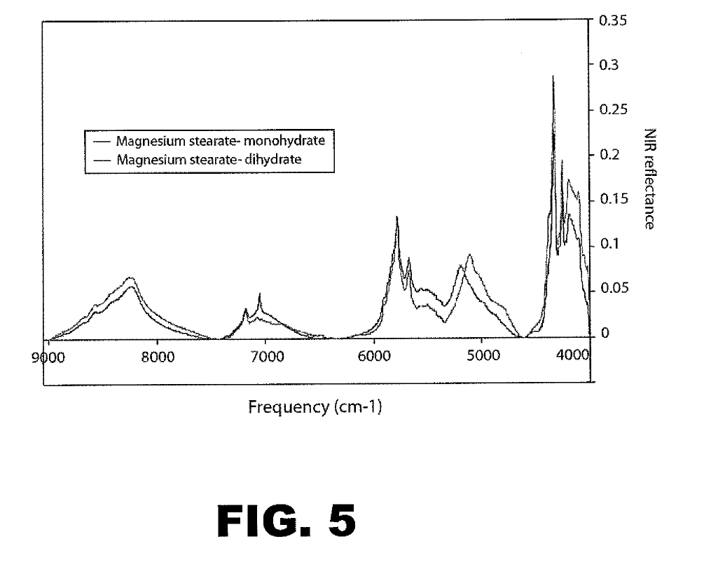

Near infrared spectroscopy analysis (NIR analyzer, Thermo Fisher Scientific, Waltham, Mass.) was conducted using standard mixtures having known compositions of MgSt monohydrate and dihydrate prepared from 92.0% MgSt-M and 95.4% MgSt-D stock material. The NIR spectra of the MgSt hydrates are shown in FIG. 5.

Conclusions. The physicochemical analysis presented in Examples 1-3 using scanning electron microscopy, powder X-ray diffraction, near-infrared spectroscopy, differential scanning calorimetry, particle-size analysis, and thermogravimetry revealed that there are discernable differences between the MgSt monohydrate and MgSt dihydrate.

### Examples 4-8

### Influences of MgSt Hydrates on Blends and Tablets

The influence of MgSt on powder lubrication and finished solid-dose products has presented significant challenges to drug manufacturing, including poor production efficiency and variability in drug disintegration and dissolution. The variation of crystalline states and their amounts in MgSt products could affect consistency in powder blending and the compressibility and quality of the resulting tablets. This example examines the influence of MgSt hydrates on blends and tablets using ternary systems comprising an active pharmaceutical ingredient with different diluent mixes (i.e., a plastically deformable-brittle diluent mix or a plastically deformable-plastically deformable diluent mix). Various ratios of diluents were used to justifiably rule out any ingredient bias that could be attributed to lubricant affinity to one diluent system. The influence of the pseudopolymorphic MgSt-M and MgSt-D on blends was profiled in real time with in-line thermal effusivity sensors during blending and lubrication steps and with an instrumented tablet press during compression. The generation of different blends is detailed in Example 4, and the difference blends are characterized in Examples 5-8.

### Example 4

### Production of Blends and Tablets

The active pharmaceutical ingredient was acetaminophen, USP (APAP, Mallinckrodt) and the diluents were microcrystalline cellulose (MCC; Avicel PH 101, 102, FMC Biopolymer, Philadelphia, Pa.); dibasic calcium phosphate, anhydrous (DCP, Encompress, JRS Pharma, Patterson, N.Y.); and lactose monohydrate, spray-dried (LAC, Spectrum Chemicals, N.J.). All materials were used as received and delumped before mixing.

Two ratios of binary diluents were used (75:25 and 50:50) for each of MCC:DCP and MCC:LAC. These binary diluents constituted desirable solid dosage formulation systems in that the physical characteristics are unique. In the case of the MCC:DCP system, the physical interaction between a plastically deformable material (MCC) and an abrasively brittle material (DCP) with distinct particle—particle shapes was deemed informative. Moreover, using two grades of MCC with different particle sizes (Avicel PH 101 had a nominal mean particle size of 50 μm, and Avicel PH 102 had a nominal mean particle size of 100 μm) could present additional valuable information through their blending behavior. With respect to the MCC:LAC system, two plastically deformable diluents with distinct particle-particle morphology would be another opportunity to elucidate the influence of MgSt in such widely used pharmaceutical combinations.

APAP was used at concentrations of 1.25, 2.5, and 5.0% w/w, MgSt was used at concentrations of 0.3, 0.5, and 1.0% w/w. The experimental design was a modified Plackett-Burman fractional factorial having two levels with two center points. Eleven batches, each at 10-kg batch size, were blended in a 1-ft³ twin-shell blender (Patterson-Kelley, Stroudsburg, Pa.). This fractionalization allowed for a reduction of input variables or factors with the benefit of identifying the key factor variables that affected product quality. The design also enabled the evaluation of main effects aliased with two-way interactions, Tables 2 and 3 show the designs and independent variables (factors). Results from the experimental design provided information for optimization of the study (see Table 2). Subsequently, six optimization batches were processed to substantiate the preliminary findings from the 11 batch runs (see Table 3). The dependent variables (responses) included ejection force and total compression force (precompression and main compression forces).

TABLE 2
Experimental Design.

| Batch | MCC | MgSt | Lube Time | | APAP | MCC/DCP |
|---|---|---|---|---|---|---|
| No. | (μm) | Type | (min) | % MgSt | (%) | ratio |
| 1 | 50 | MgSt-M | 10 | 0.3 | 5.0 | 75/25 |
| 2 | 50 | MgSt-D | 4 | 0.3 | 5.0 | 50/50 |
| 3 | 100 | MgSt-M | 4 | 0.3 | 2.5 | 50/50 |
| 4 | 100 | MgSt-D | 10 | 0.3 | 2.5 | 75/25 |
| 5 | 100 | MgSt-M | 7 | 0.5 | 1.25 | 50/50 |
| 6 | 50 | MgSt-D | 7 | 0.5 | 1.25 | 50/50 |
| 7 | 50 | MgSt-M | 4 | 1.0 | 2.5 | 75/25 |
| 8 | 100 | MgSt-M | 10 | 1.0 | 1.25 | 75/25 |
| 9 | 100 | MgSt-M | 4 | 1.0 | 1.25 | 50/50 |
| 10 | 50 | MgSt-D | 10 | 0.3 | 5.0 | 75/25 |
| 11 | 50 | MgSt-D | 4 | 1.0 | 2.5 | 75/25 |

TABLE 3
Optimized Design.

| Batch | MgSt | Lube Time | | APAP | MCC_{50 μm}/LAC |
|---|---|---|---|---|---|
| No. | Type | (min) | % MgSt | (%) | ratio |
| 12 | MgSt-M | 4 | 1.0 | 1.25 | 75/25 |
| 13 | MgSt-M | 2, 4, | 1.0 | 1.25 | 75/25 |
| | | 8, 12, 16 | | | |
| 14 | MgSt-D | 4 | 1.0 | 1.25 | 75/25 |
| 15 | MgSt-D | 2, 4, | 1.0 | 1.25 | 75/25 |
| | | 8, 12, 16 | | | |
| 16 | MgSt-M | 4 | 1.0 | 1.25 | 50/50 |
| 17 | MgSt-D | 4 | 1.0 | 1.25 | 50/50 |

Prelubrication blend uniformity was predicted using multiple effusivity sensors fitted to the blender as described by Okoye et al. (2006, ISPE News Magazine 3(3):4-8). Prelubrication and postlubrication blend uniformity samples were collected using a sampling thief (Globe Pharma, New Brunswick, N.J.) for comparative analysis. Blend samples were analyzed for the APAP assay with an internally validated high-performance liquid chromatography (HPLC) method.

The blends were compressed using a 10-station instrumented tablet press (Natoli Engineering, St Louis, Mo.) with 0.4375-in. standard round, concave tooling. All tablets were compressed to target hardness of 8.0 kp using a hardness tester (Pharmatest, Piscataway, N.J.), and target weight of 500 mg was measured using a bench scale (Sartorius, New York, N.Y.). Tablet press speed was maintained at 17 rpm. Tablet friability limit was set at not more than (NMT) 0.8% using a friability tester (Pharmatest).

Lubricant performance and influence on the tablets' physical attributes were evaluated on the basis of the main compression force, precompression force, ejection force, and tablet knock-off using a real-time data acquisition tool (Natoli Engineering, St. Louis, Mo.). In vitro dissolution studies were conducted according to USP Method, and a similarity factor, f₂, was derived for comparative analysis. Data analysis was conducted using a statistical tool (“Minitab,” Minitab Inc., State College, Pa.).

### Example 5

### Predicted Blend Homogeneity and Assay

A baseline run was conducted using neat microcrystalline cellulose, NF (MCC) to enable the effusivity sensors to predict homogeneity via in-line and real-time measurements. The placebo material was blended for a specified duration, and the synchronization pulse with baseline was established for the effusivity sensors.

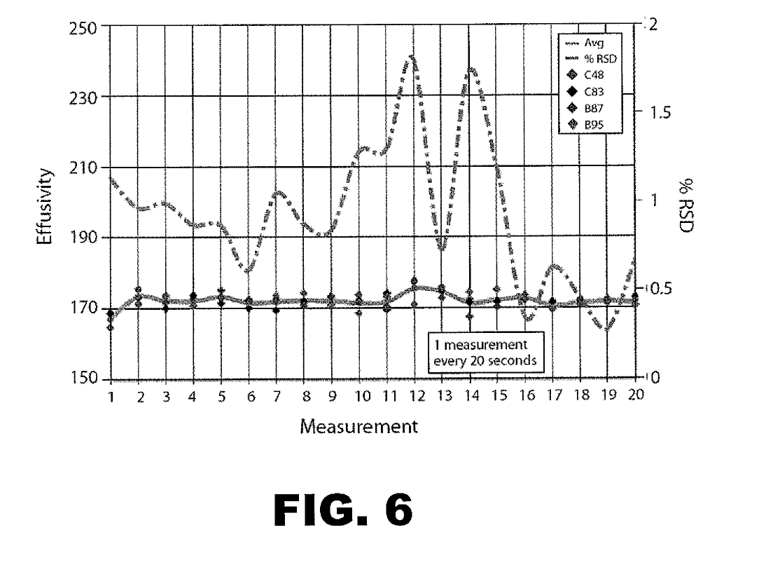

The prelubrication homogeneity of the blends was determined on the basis of real-time analysis conducted with Effusivity Sensor Package software (ESP, Mathis Instruments, Fredericton, Canada). The system synchronization enabled the sensors to dynamically obtain a real-time data stream from the rotating blender (see FIG. 6). Effusivity sensors monitor the blending of powder particles on the basis of the heat-transfer properties of the composite powder mixture. An explanation for this sensitivity to material blending or mixing is related to the ability of these powder particles or ingredients to mix in various proportions as they attend homogeneity. This is analogous to a move from “divergence” toward “convergence” or uniformity. The selection of any empirical uniformity value may depend on several factors, including the nature of materials, type of process, and individual effusivity values of the ingredients. It is well established that low relative standard deviation (RSD) in a blending process signifies uniformity in mixing or blending. End-point prediction with effusivity was based on the combination of stable average effusivity and a decaying RSD.

Blend samples were collected using a sample thief from each batch at the end of prelubrication and postlubrication blending. Blend samples of about two times the unit-dose (500 mg×2=1000 mg) were tested based on an internally validated HPLC method for APAP. The mobile phase was a mixture of methanol and water with a flow rate of 1.0 mL/min and detection at 280 nm.

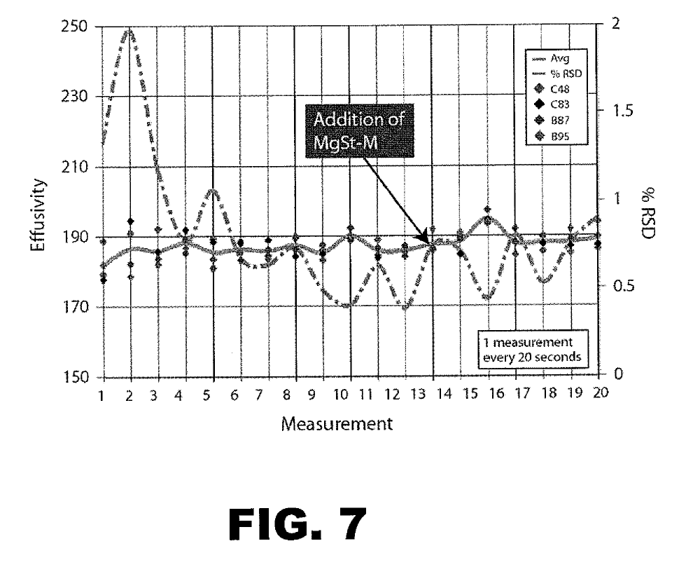

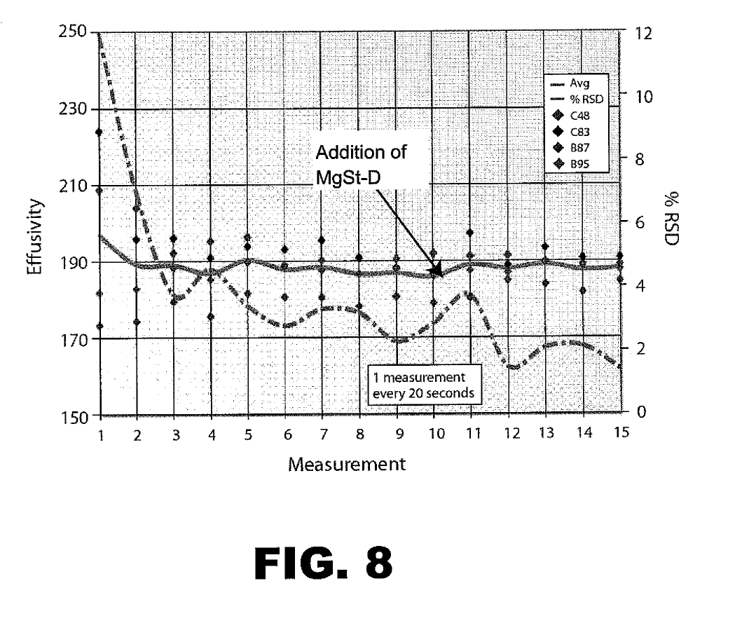

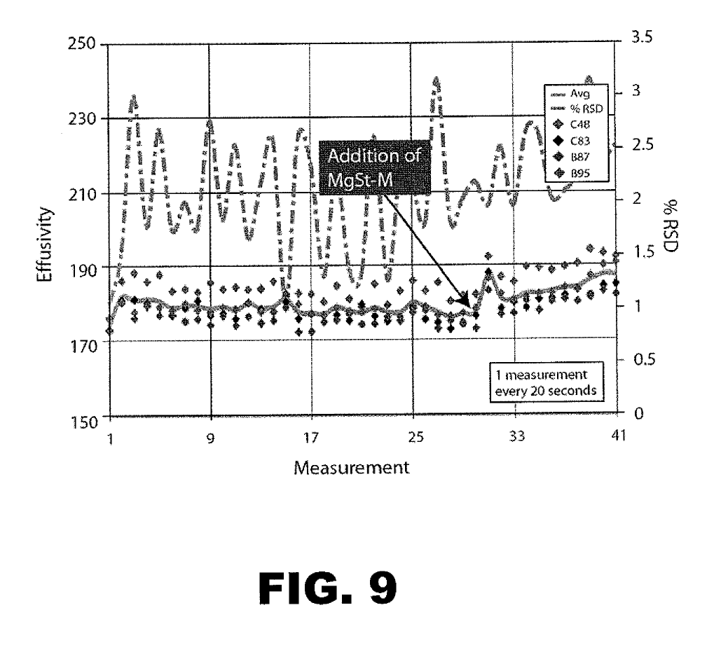

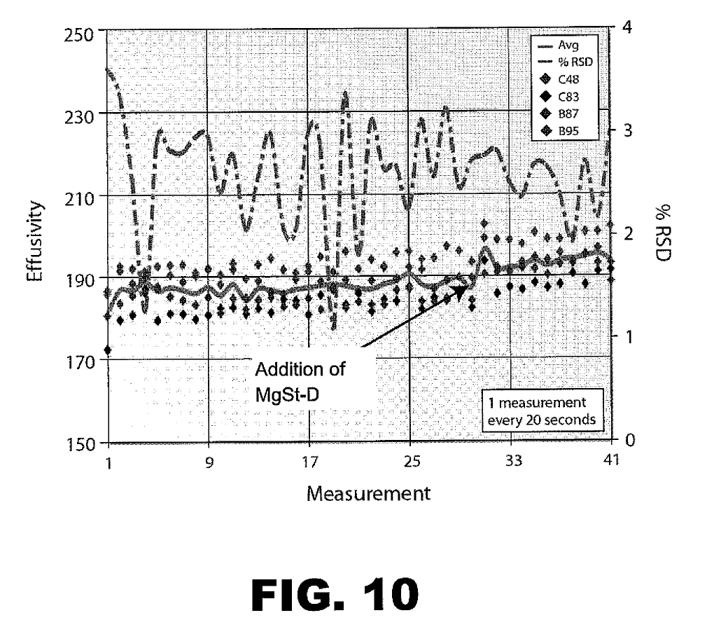

The profile of the baseline run with neat MCC is depicted in FIG. 6. Results show an average effusivity for the placebo material of 171 Ws^{0.5}/m²K (n=20), with RSD=0.7%. FIGS. 7 (Batch 7) and 8 (Batch 11) show the effusivity profiles for ternary blends of MCC, DCP (75:25), and 2.5% w/w APAP lubricated with 1.0% w/w MgSt-M and MgSt-D, respectively. FIGS. 9 (Batch 12) and 10 (Batch 14) show the effusivity profiles for ternary blends of MCC, LAC (75:25), and 1.25% w/w APAP lubricated with 1.0% w/w MgSt-M and MgSt-D, respectively.

Table 4 shows the results of the physical and chemical testing for the blends. Blend results indicate that the prelubrication end-points as predicted by effusivity sensors gave good correlation to the blend assay from HPLC analysis. Blend uniformity results for Batch 12, after 4 min of lubrication with MgSt-M, however, show a mean blend uniformity assay of 109.0%, with a failing RSD of 23.5%. Conversely, Batch 14 lubricated with MgSt-D shows an acceptable mean blend uniformity assay of 94.8% with an RSD of 1.6%.

TABLE 4
Blend Uniformity and Effusivity Results.

| | Batch 7 | Batch 11 | Batch 12 | Batch 14 |
|---|---|---|---|---|
| Prelube blend | 100.3 | 99.3 | 98.8 | 100.8 |
| uniformity assay | | | | |
| % RSD on assay | 1.5 | 2.0 | 1.2 | 3.1 |
| Postlube blend | 97.5 | 97.0 | 109.0 | 94.8 |
| uniformity assay | | | | |
| % RSD on post lube | 1.7 | 1.4 | 23.5* | 1.6 |
| at 4 min | | | | |
| Prelube end-point | 188.4 | 188.5 | 178.0 | 188.0 |
| effusivity | | | | |
| % RSD in prelube | 2.3 | 2.2 | 2.0 | 2.5 |
| effusivity | | | | |
| % MgSt used | 1.0 | 1.0 | 1.0 | 1.0 |
| MgSt type | MgSt-M | MgSt-D | MgSt-M | MgSt-D |
| MCC_{50 μm}:DCP ratio | 75:25 | 75:25 | | |
| MCC_{50 μm}:LAC ratio | | | 75:25 | 75:25 |
| % APAP (w/w) | 2.50 | 2.50 | 1.25 | 1.25 |

*Batch 12 lubricated with 1.0% MgSt-M failed on RSD.

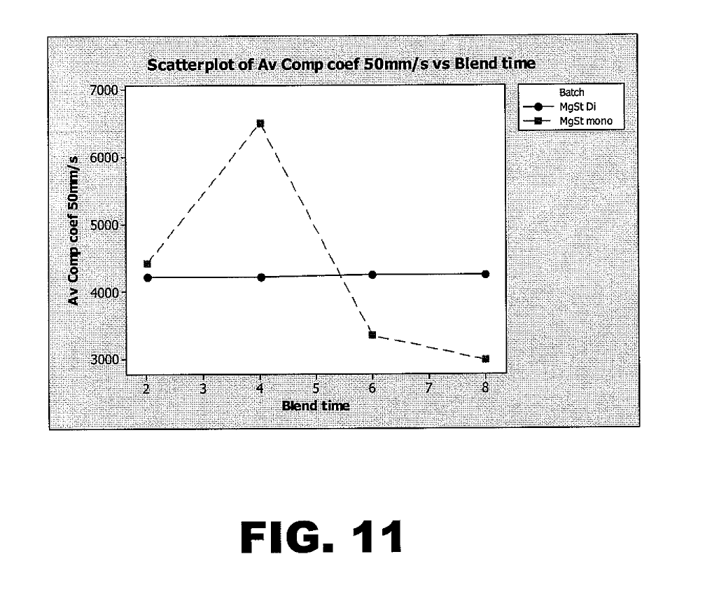

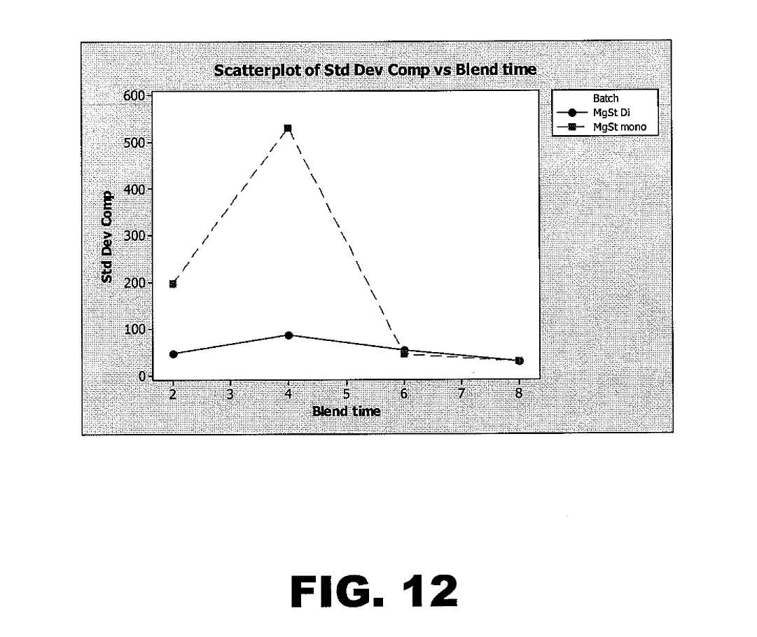

Scatterplot analysis of the average compression coefficient 50 mm/s as a function of blend (i.e., lubrication) time revealed that dihydrate blends differed dramatically from monohydrate blends at 4 min (see FIG. 11). Similarly, scatterplot analysis of the compression standard deviation vs. blend time showed dihydrate blends differed dramatically from monohydrate blends at 4 min (see FIG. 12).

### Example 6

### The Influence of Lubrication and MgSt Type on Blend Integrity

The influence of MgSt type and concentration on effusivity was analyzed using one-way analysis of variance (ANOVA). Analysis of the change (delta) in average effusivity between prelubrication and postlubrication blends containing MgSt-M and MgSt-D, using Tukey's paired comparison at 95% confidence limit, shows statistical significance of p<0.05 (see Table 5). The pair wise comparison is indicative of the differing influence attributable to the distinct hydrate forms of the lubricant.

TABLE 5
The Influence of MgSt Type on Blend Integrity.

| | | | | | Responses | | |
|---|---|---|---|---|---|---|---|
| | | | Factors | | Delta | Total comp | Eject |
| | Diluent | Diluent | MgSt | | effusivity* | force | force |
| Batch | system | ratio | type | % MgSt | (Ws^{0.5}/m²K) | (kN) | (N) |
| 1 | MCC:DCP | 75:25 | Mono | 0.3 | 10 | 12 | 11 |
| 7 | MCC:DCP | 75:25 | Mono | 1.0 | 7 | 13.1 | 11 |
| 10 | MCC:DCP | 75:25 | Di | 0.3 | 3 | 12.1 | 10 |
| 11 | MCC:DCP | 75:25 | Di | 1.0 | 3 | 11.2 | 9 |
| 12 | MCC:LAC | 75:25 | Mono | 1.0 | 10 | 10.3 | 8.6 |
| 14 | MCC:LAC | 75:25 | Di | 1.0 | 6 | 9.3 | 6.7 |
| 16 | MCC:LAC | 50:50 | Mono | 1.0 | 16 | | |
| 17 | MCC:LAC | 50:50 | Di | 1.0 | 10 | | |

*Delta effusivity is the change in average effusivity between post- and pre-lubrication. All batches were lubricated for 4 min.

Results from the blending studies, as profiled by the in-line effusivity sensors, also showed that when ternary systems containing MCC-DCP and MCC-LAC as diluents were lubricated with MgSt-M and MgSt-D, the delta effusivity values were higher for the blends containing MgSt-M. (Compare Batch 7 and Batch 12 versus Batch 11 and Batch 14 in Table 5). These results indicate the ternary systems containing MgSt-D showed less degree of densification for both MCC-LAC and MCC-DCP diluent systems before and after lubrication.

Similarly, with the same 75:25 diluent ratio, MCC-DCP blends lubricated with MgSt-M exhibited 2-3 times more densification than MgSt-D (Batch 7 versus Batch 11). Also MCC-LAC blends with a 75:25 ratio, when lubricated with MgSt-M, showed about 1.6 times more densification than blends with MgSt-D (Batch12 versus Batch 14). Moreover, within the MCC-LAC diluent system, the 50:50 diluent ratio tended to show higher delta effusivity than the 75:25 ratio. (Batch 12 versus Batch 16, and Batch 14 versus Batch 17). This result could be attributed to the increasing contribution of lactose in the formulation, particle-particle interaction, and diluent-type sensitivity to the influence of MgSt. Although the mechanism of the densification may not be fully understood, it is believed that the finer particles of the lubricant tend to displace the air pockets between larger particles and occupy the interstices with a resultant more densely packed powder mixture, Such particulate packing, presumably a result of MgSt addition, could disturb the established blend uniformity.

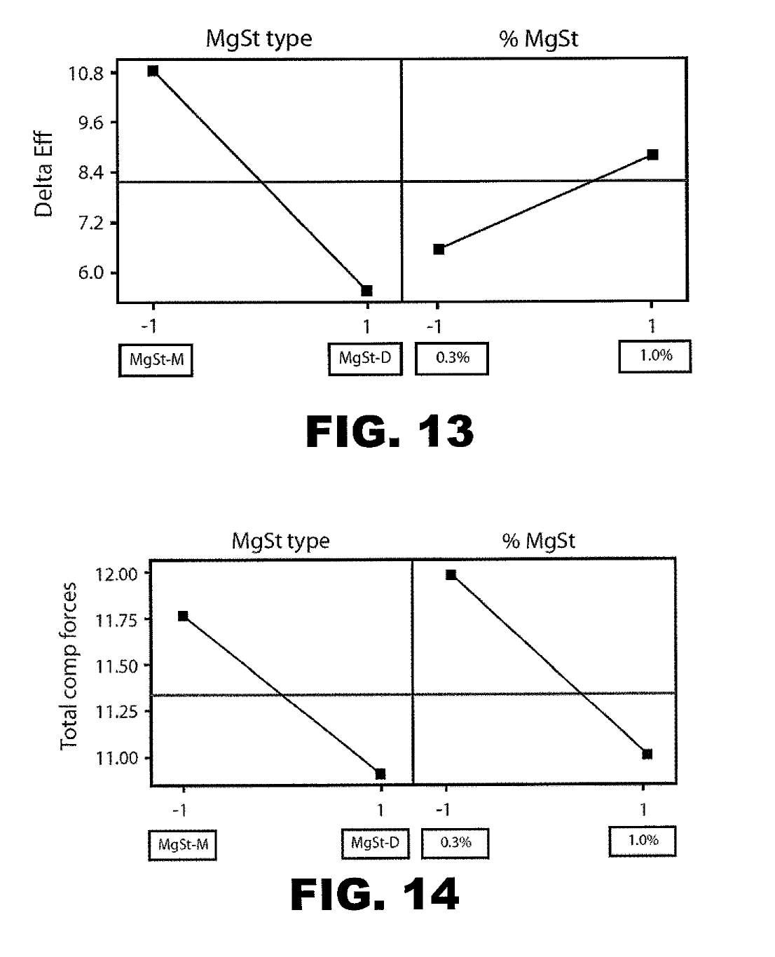

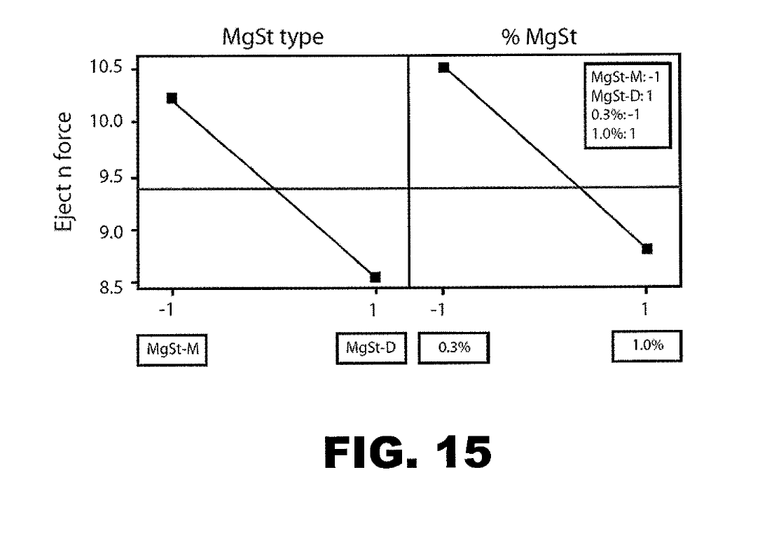

FIG. 13 shows that the delta on average effusivity was greater for MgSt-M than for MgSt-D. In addition, the results indicate that the average total compression forces and ejection force were lower for MgSt-D than the MgSt-M (see Table 6 and FIGS. 14 and 15).

### Example 7

### Physical Attributes and Content Uniformity of the Tablets

Compression of the batches was conducted using a 10-station instrumented press (Natoli Engineering). Compression parameters were monitored based on constant (target) tablet weight (500 mg) and hardness (8 kp). Based on the fact that MgSt type, percentage of MgSt, and lubrication time differed in the batches, the effects of these variables on precompression force, main compression force, ejection force, knock-off force, and tablet friability were monitored or measured to evaluate the level of such influence. Additional influence was also expected from the differing percentage of APAP and diluents. An attempt was made to statistically analyze such influence to understand the main effects and interactions.

Using a stratified sampling method, tablets were collected at intervals during the compression runs. Content uniformity was conducted using an internally validated HPLC method for APAP. Mean assay and % RSD for 10 tablets were determined.

The data in Table 6 show the compression batches containing MCC-DCP and MCC-LAC binary diluents systems. Results show that except for Batch 8, all batches gave acceptable results for content uniformity. The mean assay for 10 tablets for Batch 8 was 96.9%. The % RSD was 7.9, however, which is much higher than the acceptable limit. Batch 8 was lubricated with 1.0% of MgSt-M for 10 min. This result implies that an extended period of lubrication could affect the tablets' content uniformity.

The tablet characteristics shown in Table 6 depict some distinct effects in the total compression forces, ejection force, and tablet knock-off between the blends lubricated with different pseudopolymorphic forms of MgSt. These differences in tableting forces appear to be evident under similar formulations and with preset target ranges for tablet weight and hardness. So long as the preblend components of the formula are comparable, the anticipated variables would include percentage of MgSt and duration of lubrication. These two variables tend to influence the compressibility, tablet ejection, and knock-off. The efficiency of a lubricant during a tableting operation hinges on its ability to facilitate tablet release postcompression. The amount of such lubricants, however, combined with the duration of lubrication often influence the forces acting on the upper and lower punches.

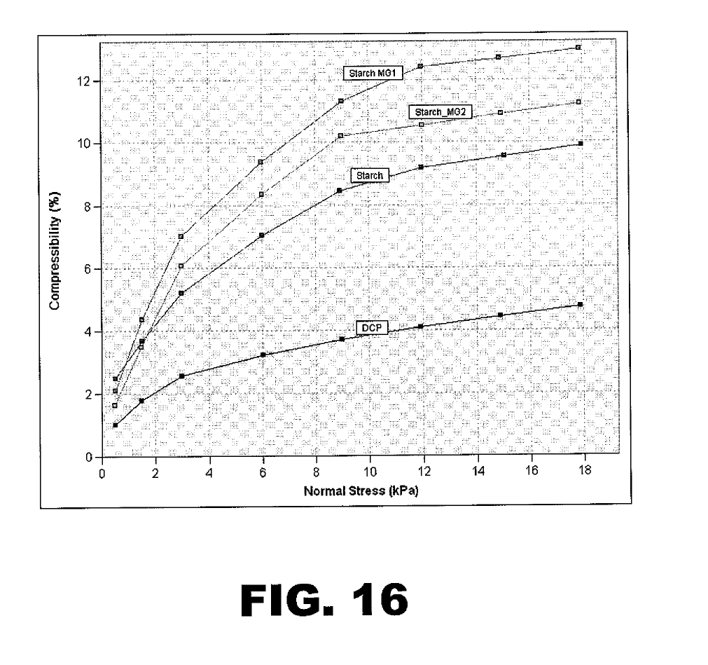

A powder rheometer (FT4, Freeman Technology, Worcestershire, UK) was used to measure compressibility of tablets that had starch as a diluent. FIG. 16 shows that tablets with either MgSt hydrate had greater compressibility than those without.

TABLE 6
Physical and Chemical Results for Tablets.

| | Batch Number | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 14 |
| Average tablet weight | 493.9 | 508.0 | 504.4 | 491.0 | 499.4 | 496.1 | 507.7 | 502.4 | 497.5 | 497.4 | 491.2 | 497.0 | 497.0 |
| (mg) | | | | | | | | | | | | | |
| RSD on tablet weight | 0.4 | 0.6 | 1.3 | 0.4 | 1.3 | 1.6 | 0.5 | 0.9 | 0.5 | 0.3 | 0.9 | 0.5 | 0.6 |
| (n = 10) | | | | | | | | | | | | | |
| Average tablet | 8.4 | 8.1 | 8.0 | 8.0 | 8.1 | 8.1 | 8.1 | 8.5 | 8.3 | 8.5 | 8.3 | 8.2 | 8.1 |
| hardness (kp) | | | | | | | | | | | | | |
| MgSt type | Mono | Di | Mono | Di | Mono | Di | Mono | Mono | Di | Di | Di | Mono | Di |
| MCC:DCP ratio | 75:25 | 50:50 | 50:50 | 75:25 | 50:50 | 50:50 | 75:25 | 75:25 | 50:50 | 75:25 | 75:25 | | |
| MCC:LAC ratio | | | | | | | | | | | | 75:25 | 75:25 |
| Lubrication time (min) | 10 | 4 | 4 | 10 | 7 | 7 | 4 | 10 | 4 | 10 | 4 | 4 | 4 |
| % MgSt | 0.3 | 0.3 | 0.3 | 0.3 | 0.5 | 0.5 | 1.0 | 1.0 | 1.0 | 0.3 | 1.0 | 1.0 | 1.0 |
| MCC particle size (μm) | 50 | 50 | 100 | 100 | 100 | 50 | 50 | 100 | 100 | 50 | 50 | 50 | 50 |
| APAP (%) | 5.0 | 5.0 | 2.5 | 2.5 | 1.25 | 1.25 | 2.5 | 1.25 | 1.25 | 5.0 | 2.5 | 1.25 | 1.25 |
| Precomp force (kN) | 3.75 | 2.81 | 3.12 | 3.87 | 3.06 | 4.07 | 4.12 | 4.67 | 3.67 | 3.77 | 3.59 | 4.42 | 4.40 |
| Main compression (kN) | 8.24 | 12.9 | 13.61 | 7.87 | 11.71 | 12.6 | 8.99 | 11.47 | 18.4 | 8.36 | 7.63 | 5.86 | 4.92 |
| Ejection (N) | 10.6 | 24.3 | 16.4 | 10.7 | 14.3 | 15.9 | 10.9 | 13.0 | 19.4 | 10.4 | 9.3 | 8.6 | 6.7 |
| Total comp force (kN) | 11.99 | 15.71 | 16.73 | 11.74 | 14.77 | 16.67 | 13.11 | 16.14 | 22.07 | 12.13 | 11.22 | 10.3 | 9.3 |
| Mean content | 101.6 | 97.2 | 103.9 | 104.4 | 99.8 | 97.2 | 99.1 | 96.9 | 98.5 | 98.3 | 96 | 99.1 | 98.2 |
| uniformity (%) | | | | | | | | | | | | | |
| % RSD on content | 2.1 | 4.3 | 1.7 | 3.0 | 2.4 | 1.9 | 2.4 | 7.9* | 1.9 | 3.2 | 1.7 | 1.5 | 2.0 |
| uniformity | | | | | | | | | | | | | |

*Batch 8 lubricated with 1.0% MgSt-M failed on RSD.

Based on a Plackett-Burman design, % MgSt, MCC-DCP ratio, MSS particle size, % APAP, and lubrication time were evaluated for their influence on ejection force and total compression force. Using the method of least squares, regression models were developed for the total force (precompression and main compression forces) and the ejection force to elucidate the influence of the lubricants on the compression process.

Table 7 shows the regression analysis for the ejection force. Based on the tablet physical results, the diluent ratio had the greatest influence on ejection force (p<0.005). The data also showed that the % API and % MgSt in the formulation had second- and third-highest influence on tablet ejection based on the coefficient at p<0.005. Overall, R² (indicating the linearity of the regression) was 0.9250, suggesting that the selected model design was appropriate.

TABLE 7
Regression Analysis for Ejection Force.

| Predictor | Coefficient | SE coefficient | T | P |
|---|---|---|---|---|
| Constant | 16.352 | 0.880 | 18.470 | 0.000 |
| % MgSt | 4.477 | 1.400 | 3.200 | 0.024 |
| Lube time | 2.775 | 1.060 | 2.610 | 0.048 |
| Particle size | 1.837 | 0.665 | 2.760 | 0.040 |
| Diluent ratio | −6.537 | 1.007 | −6.490 | 0.001 |
| % APAP | 5.858 | 1.545 | 3.790 | 0.013 |
| S = 1.1813 | R² = 92.5% | R² (adj) = 83.6% | | |

In addition, the regression model for the total forces (precompression and main-compression forces) as depicted in Table 8 shows that the diluent ratio also had the highest influence on combined compression forces (p<0.005). The second and third highest-ranking responses, based on the coefficient at p<0.005, were the percentage of MgSt and the type of MCC, respectively. The model shows a linearity, R²=0.9110. These regression models show that the influence of the diluent ratio, percentage of MgSt, percentage of API, and type of MCC, if held constant, could offer some insights into the subtle characteristics of other factors such as the type of MgSt and the duration of lubrication. As such, an optimized design was constructed to keep these factors the same and minimize their influence to fully elucidate the presence (or absence) of influence of differing MgSt hydrates.

TABLE 8
Regression Analysis for Total Force (Precompression and Main).

| Predictor | Coefficient | SE coefficient | T | P |
|---|---|---|---|---|
| Constant | 1622.480 | 66.630 | 24.350 | 0.000 |
| % MgSt | 313.800 | 105.400 | 2.980 | 0.031 |
| Lube time | 147.240 | 79.960 | 1.840 | 0.125 |
| Particle size | 176.950 | 50.070 | 3.530 | 0.017 |
| Diluent ratio | −355.840 | 75.750 | −4.700 | 0.005 |
| % APAP | 216.200 | 116.200 | 1.860 | 0.122 |
| S = 136.5 | R² = 91.1% | R² (adj) = 80.3% | | |

Two experiments were conducted using a binary diluent system of MCC (50 μm particle size) and LAC at the ratio of 75:25, with APAP as the active ingredient at 1.25% w/w concentration. With the level of MgSt at 1.0% w/w, the influence of lubrication on blend uniformity assay was monitored at 2-, 4-, 8-, 12-, and 16-min time points. Results show that Batch 13 lubricated with MgSt-M gave failing % RSD on blend uniformity assay at 2-min (36.0%) and 4-min (8.1%) time points (see Table 9). Results at the 8-, 12-, and 16-min time points, however, were acceptable. For Batch 15 lubricated with MgSt-D, the results at all the time points were acceptable. Although no reason was found for the failed results for Batch 13, the influence of the MgSt type on a uniformity blend could not be ruled out.

TABLE 9
Postlubrication of MCC-LAC Binary Diluent Systems.

| | Batch 13 | Batch 15 |
|---|---|---|
| | (MgSt-M) | (MgSt-D) |
| Prelube | | |
| Mean | 92.6 | 98.8 |
| RSD | 3.2 | 2.7 |
| Postlube blend uniformity (%) | | |
| 2 min | | |
| Mean | 98.7 | 94.9 |
| RSD | 36.08 | 1.7 |
| 4 min | | |
| Mean | 92.0 | 95.5 |
| RSD | 8.18 | 2.0 |
| 8 min | | |
| Mean | 95.6 | 98.1 |
| RSD | 2.7 | 3.8 |
| 12 min | | |
| Mean | 96.8 | 95.7 |
| RSD | 3.3 | 1.5 |
| 16 min | | |
| Mean | 97.1 | 97.1 |
| RSD | 0.9 | 2.1 |

*Batch 13 failed RDS at 2 and 4 min lubrication.

### Example 8

### In Vitro Dissolution of the Tablets

In vitro dissolution studies were performed using an USP Type 2 dissolution apparatus at 50 rpm. The dissolution media consisted of 900 mL degassed purified water, USP, maintained at 37° C.±0.5° C. A 5-mL aliquot was withdrawn at intervals of 5, 10, 15, and 30 min. Drug content was determined by HPLC at 280 nm. All dissolution tests were conducted in triplicate. The similarity was determined by the model independent approach using a similarity factor (f₂) as described in the FDA Guidance for Industry: Dissolution Testing of Immediate Release Solid Oral Dosage Forms (1997; FDA, Rockville, Md.). The similarity factor (f₂) is defined as follows:

$${f}_{2} = 50 \times log [ {[ 1 + ( \frac{1}{n} ) {\sum }_{t = 1}^{n} {( {R}_{t} - {T}_{t} )}^{2} ]}^{- 0.5} \times 100 ]$$

in which Rₜ and Tₜ are the average percentage of drug dissolved at each sampling time for reference (R) and the test (T) preparations, respectively, and n is the number of samples. An f₂ value between 50 and 100 suggests that the two dissolution profiles are similar.

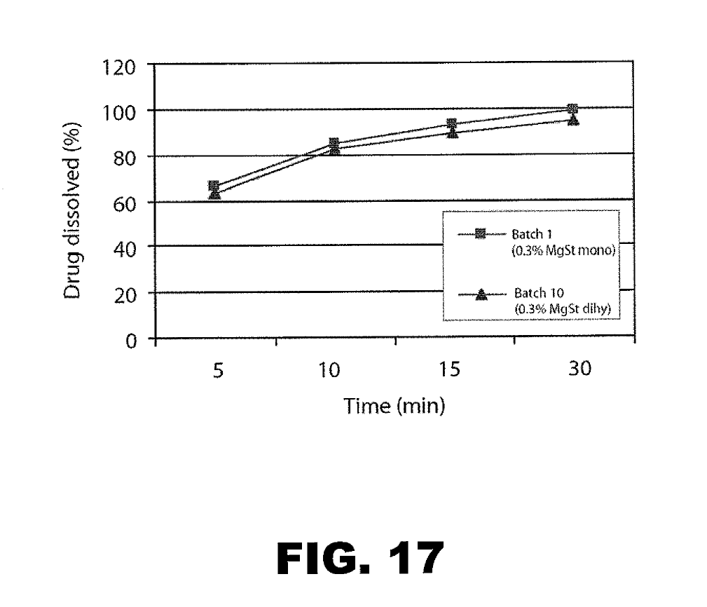

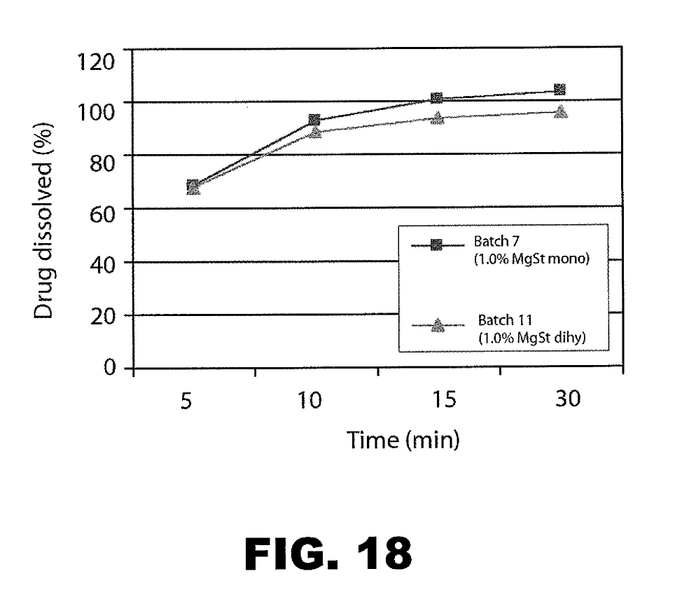

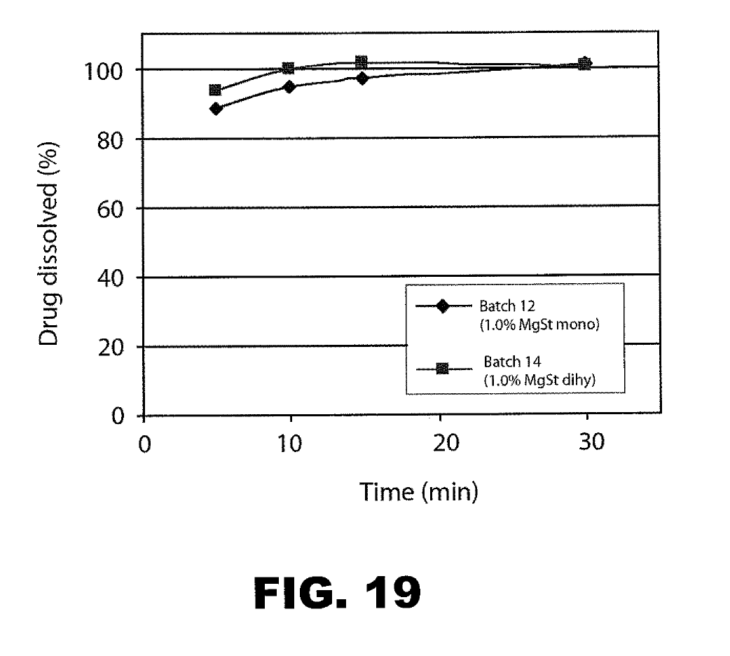

In vitro dissolution was conducted on six tablets from each of batches 1, 7, 10, 11, 12, and 14. These batches were lubricated with different amounts of MgSt-M and MgSt-D ranging from 0.3% to 1.0%. FIG. 17 shows results from Batch 1 and Batch 10 lubricated for 10 min with 0.3% MgSt-M and MgSt-D, respectively. FIG. 18 shows results of Batch 7 and Batch 11 lubricated for 4 min with 1.0% MgSt-M and MgSt-D, respectively. And FIG. 19 shows results from Batch 12 and Batch 14 lubricated for 4 min with 1.0% MgSt-M and MgSt-D, respectively. Results of the in vitro dissolution suggest that all batches met the criteria for similarity (f₂ between 50 and 100) as stipulated in the FDA manual (see FIGS. 14-16 and Table 10). These results indicate that finished products containing the dihydrate polymorph could provide comparable quality to those containing the monohydrate form.

TABLE 10
Similarity factor (f₂) for (MCC-DCP) Tablets with
Different MgSt Types.

| | Drug dissolved (%) | | | |
|---|---|---|---|---|
| | Batch 1 | Batch 10 | Batch 7 | Batch 11 |
| Time (min) | (MgSt-M) | (MgSt-D) | (MgSt-M) | (MgSt-D) |
| 5 | 66.2 | 63.6 | 68.8 | 68.2 |
| 10 | 85.3 | 82.6 | 93 | 88.3 |
| 15 | 92.8 | 89.8 | 100.5 | 93.6 |
| 30 | 99.2 | 95 | 103.6 | 95.8 |

| | |
|---|---|
| f₂ comparison = 74 | f₂ comparison = 62 |

Conclusions. The lubrication of direct-compressible blends with the hydrates of MgSt has presented evidence of differences in the effects these hydrates could have on blend homogeneity and tablet compression. In-line effusivity sensors predicted blend uniformity in all prelubrication blends down to 1.25% w/w of active pharmaceutical ingredient in the formulations. In addition, the in-line effusivity sensors suggested that lubricating blends with the monohydrate form could cause greater disturbance in blend particle arrangement and densification than the dihydrate form under similar process conditions.

Finally, compression results showed that blends lubricated with MgSt-M required higher total-compression forces, ejection force, and knock-off force than those with MgSt-D. Similarity comparison based on the f₂ factor, as conducted on finished products, indicates that the blends lubricated with MgSt-D compared well with those containing MgSt-M.

What is claimed is:

1. Use of magnesium stearate dihydrate for lubrication of a solid material by combining the solid material with a lubricant composition comprising at least 40% by weight of magnesium stearate dihydrate.

2. The use of claim 1, wherein the lubricant composition comprises at least 90% by weight of magnesium stearate dihydrate; and less than about 5% by weight of magnesium stearate monohydrate.

3. The use of any of claims 1 to 2, wherein the magnesium stearate dihydrate is a polymorph comprising stearic acid and palmitic acid.

4. The use of claim 3, wherein the weight ratio of stearic acid to palmitic acid is 2:1; the average diameter of the magnesium stearate dihydrate polymorph is from about 10 to about 20 microns; and the amount of magnesium stearate dihydrate combined with the solid material is from about 0.1% to about 20% by weight of the sum total of solid material and magnesium stearate dihydrate.

5. The use of any of claims 1 to 4, wherein the solid material is a form of matter selected from the group consisting of a powder, a bead, a granule, a crystal, a particle, an encapsulated liquid, an encapsulated semi-solid material, and an encapsulated solid material.

6. The use of any of claims 1 to 5, wherein the solid material is at least one ingredient of a product selected from the group consisting of a food product, a nutrition product, a cosmetic product, and a paint product.

7. A food product, the food product comprising an edible material and a lubricant composition comprising at least 40% by weight of magnesium stearate dihydrate.

8. The food product of claim 7, wherein the lubricant composition comprises at least 90% by weight of magnesium stearate dihydrate and less than about 1% by weight of magnesium stearate monohydrate; and the concentration of magnesium stearate dihydrate is from about 1% to about 2% by weight of the total weight of the food product.

9. The food product of any of claims 7 to 8, wherein the magnesium stearate dihydrate is a polymorph comprising stearic acid and palmitic acid, and the weight ratio of stearic acid to palmitic acid is 2:1.

10. The food product of any of claims 7 to 9, wherein the edible material is selected from the group consisting of a salt, a sugar, a flour, a starch, a leavening agent, a dry blend mix, a cereal product, a snack product, a nutrition bar, and a baked good.

11. A dry powder product, the product comprising a dry powder phase, a liquid binder phase, and a lubricant composition comprising at least 40% by weight of magnesium stearate dihydrate.

12. The powder product of claim 11, wherein the lubricant composition comprises at least 90% by weight of magnesium stearate dihydrate and less than about 1% by weight of magnesium stearate monohydrate;

13. The powder product of any of claims 11 to 12, wherein the magnesium stearate dihydrate is a polymorph comprising stearic acid and palmitic acid, and the weight ratio of stearic acid to palmitic acid is 2:1.

14. The powder product of any of claims 11 to 13, wherein the dry powder phase comprises a powder selected from the group consisting of a mineral silicate, an organic polymer powder, a synthetic polymer powder, a silicone resin powder, and combinations thereof; the liquid binder phase is selected from the group consisting of an oil, a hydrocarbon, a liquid synthetic ester, a silicone oil, a wax, and combinations thereof; and the product is a cosmetic product selected from the group consisting of a dry foundation, a pressed powder, a loose powder, a dry blush, an eye shadow, an eye pencil; and combinations thereof.

15. The powder product of any of claims 11 to 13, wherein the product is a dry paint product further comprising a polymer selected from the group consisting of acrylic, polyester, polyether, polyurethane, and combinations thereof.
